(19) United States
(12) Patent Application Publication
(10) Pub. No.: US 2026/0171056 A1
(43) Pub. Date: Jun. 18, 2026

(54) MELODIES EMBEDDED WITH GAMMA WAVES

(71) Applicants: Naomi Fabricant, Beaufort, SC (US); Salvatore Erna, Windham, NH (US); Patrick Rowe, Reno, NV (US)

(72) Inventors: Naomi Fabricant, Beaufort, SC (US); Salvatore Erna, Windham, NH (US); Patrick Rowe, Reno, NV (US)

(21) Appl. No.: 18/981,007

(22) Filed: Dec. 13, 2024

(51) Int. Cl. G10H 1/14; A61M 21/00; G10H 1/38

(52) U.S. Cl. CPC G10H 1/14; A61M 21/00; G10H 1/383; A61M 2021/0027; G10H 2210/056; G10H 2210/335; G10H 2210/571

(57) ABSTRACT

A system and method for incorporating gamma-frequency sound waves into a melody is described. The method includes identifying a chord sequence of the melody, the melody comprising a plurality of chord sequences, and identifying a chord of the chord sequence. A root note for the chord is identified. The method includes identifying an amplitude level of the chord sequence, and selecting a gamma wave having a selected gamma wave frequency of between 15 Hz and 80 Hz, the selected gamma wave frequency selected to correspond to the root note. The selected gamma wave is embedded over the chord sequence at a gamma wave amplitude of 0.1 to 10 decibels above the chord sequence amplitude level, and the steps are repeated for one or more additional chord sequences to produce a gamma wave embedded melody.

### FIELD OF INVENTION

The invention relates to the field of music-based interventions (MBIs) and, more particularly, embedding a dynamic stream of gamma waves into melodies.

### BACKGROUND

Despite how prevalent mental health and neurological conditions are, many of these conditions still have no definitive cures, or have treatments that come with significant risks or uncertain long-term or deleterious side effects that can disrupt a patient's wellbeing and quality of life. Given the limited success of pharmacological interventions (whether it is effectiveness, side effects, or that these treatments often manage only the symptoms), there is a growing recognition that more holistic approaches are both necessary and desirable.

Recently, non-invasive (e.g., auditory and visual stimulation) gamma-frequency stimulation has been shown to reduce multiple biomarkers of dementia, provide neuroprotection, and improve cognitive function. For instance, studies show that one hour of non-invasive gamma-frequency auditory stimulation both enhanced neural activity and reduced amyloid-beta peptide levels within the auditory cortex and hippocampus. Additionally, gamma-frequency stimulation was also found to enhance cognition and memory.

A major challenge regarding the use of gamma-frequency auditory stimulation is that gamma-frequency sounds can be unpleasant, uncomfortable, or simply not interesting for humans, thus making it difficult to administer non-invasive auditory treatments for long periods of time.

One solution to overcome this challenge is to incorporate gamma-frequency sounds into music. In fact, music embedded with sub- and low-bass gamma frequencies (20 Hz to 80 Hz) has been shown to act as a “cognitive amplifier.” This approach leverages the familiar, relaxing effects of music with the cognitive-enhancing properties of low-bass frequencies, creating a tool that engages the brain in multiple ways for improved focus, memory, and mental clarity. Further, this approach has shown promise for supporting neuroplasticity and cognitive resilience, which are particularly beneficial for aging brains or individuals managing cognitive health issues. In these applications, gamma frequencies are overlaid into music using a static, steady-state method. However, this method interferes with both the music quality and the listener's experience. A dynamic approach to incorporate gamma-frequency sounds into music in a manner that is both harmonious with the existing music and retains the artistic qualities of the music and listener's experience has not been achieved.

Accordingly, a need exists for improved methods and systems for dynamically incorporating gamma frequencies into existing music while still preserving musical harmony and providing the same or additional amounts of health benefits as using traditional gamma-frequency auditory stimulation treatments.

### SUMMARY

To address the above and other deficiencies with existing gamma-frequency auditory stimulation treatments, the present invention contemplates a method and system for improved gamma-frequency auditory stimulation treatment by dynamically incorporating gamma frequencies into musical melodies while still retaining harmony with the existing music and achieving the same or additional optimal health benefits as traditional stimulation methods.

Various embodiments of a method and system for dynamically incorporating gamma frequencies are disclosed.

According to a preferred embodiment, a method for embedding gamma waves into a melody comprises selecting a melody and identifying the root notes of the melody. It further comprises identifying frequencies of gamma waves which correspond to the root notes identified and determining an amplitude level of the gamma waves.

In an embodiment, a method for incorporating gamma-frequency sound waves into a melody is described. The method comprises identifying a chord sequence of the melody, the melody comprising a plurality of chord sequences; identifying a chord of the chord sequence; identifying a root note for the chord, identifying an amplitude level of the chord sequence, selecting a gamma wave having a selected gamma wave frequency of between 15 Hz and 80 Hz, the selected gamma wave frequency selected to correspond to the root note; embedding the selected gamma wave over the chord sequence at a gamma wave amplitude of 0.1 to 10 decibels above the chord sequence amplitude level; and repeating the above steps for one or more additional chord sequences to produce a gamma wave embedded melody.

A system is further contemplated wherein one embodiment may include a Frequency Modulation Synthesizer programmed to receive melodies and comprised of several modules to embed gamma waves into the melody.

### BRIEF DESCRIPTION OF THE DRAWINGS

Certain embodiments are shown in the drawings. However, it is understood that the present invention is not limited to the methods and systems shown in the attached drawings.

FIG. 1 is a block diagram illustrating a preferred method for embedding gamma-frequency sound waves into melodies according to an embodiment of the invention.

FIG. 2 is an enhanced block diagram illustrating gamma-frequency selection process 1200 of FIG. 1 according to an embodiment of the invention.

FIG. 3 is a block diagram illustrating a system for embedding gamma-frequency sound waves into melodies according to an embodiment of the invention.

FIG. 4 is an enhanced block diagram illustrating a synthesizer module 250 of FIG. 3 according to an embodiment of the invention.

FIG. 5 is an enhanced block diagram illustrating a mixing module 280 of FIG. 3 according to an embodiment of the invention.

### DETAILED DESCRIPTION

For the purposes of promoting and understanding the principles disclosed herein, reference is now made to the preferred embodiments illustrated in the drawings, and specific language is used to describe the same. It is nevertheless understood that alternative embodiments that utilize one or more of the principles disclosed and illustrated herein are contemplated as within the scope of the present invention.

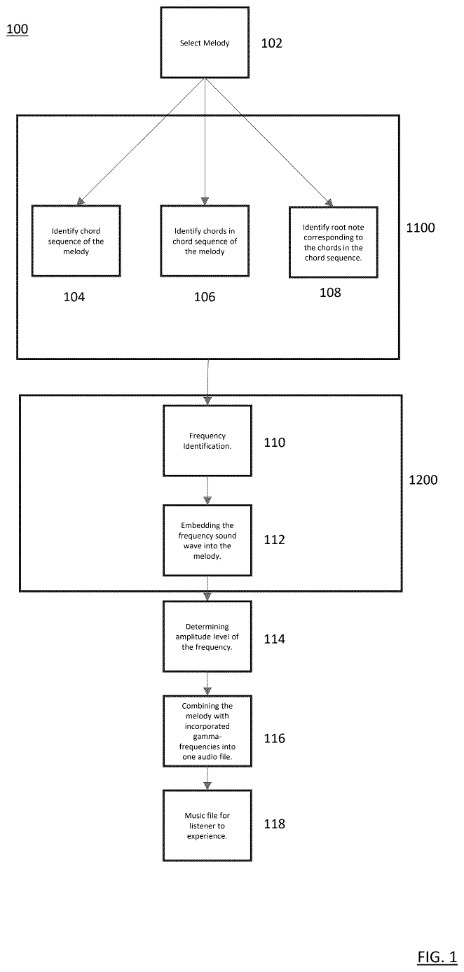

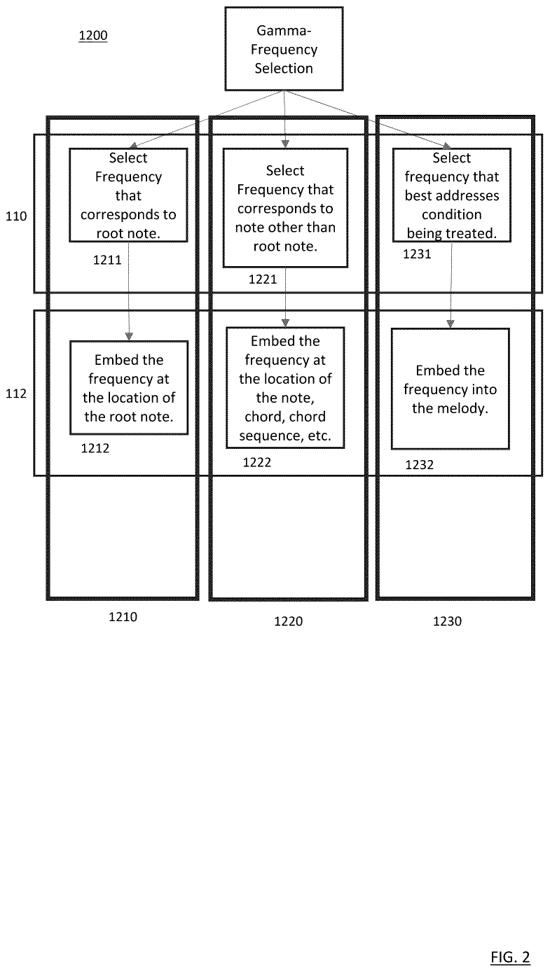

FIGS. 1-2 display a block diagram illustrating a preferred method 100 for embedding gamma-frequency sound waves into melodies. Broadly, a melody may be considered as a sequence of musical notes that a user perceives as a single entity. In a preferred embodiment, though not as a limitation, a melody may comprise a popular song or composition that a listener enjoys listening to, such as rock, jazz, R&B, instrumental pop, classical, or other popular and enjoyable genres. Such an embodiment is preferred because research shows that when individuals are exposed to music they find enjoyable, the brain's neuroplasticity is enhanced. By introducing gamma-frequency stimulation during this period, a user's cognitive processing is further amplified. Further, the brain's “heightened” and “brightened” state allows it to become more receptive to gamma-frequency stimulation.

Referring first to the embodiment of the invention depicted in FIG. 1, method 100 starts by selecting a melody 102. In an embodiment, the melody is a song or composition. In alternative embodiments (not shown), any type of audio is selected, wherein the audio may comprise ambient music, podcasts, recorded interviews, noise music, autonomous sensory meridian response (ASMR), and the like. After a melody 102 is selected, a parsing process 1100 may be performed on the melody. As illustrated in block 1100, a parsing process 1100 identifies one or more chord sequences of a melody 104, identifies one or more chords 106 in each chord sequence 104, and identifies a root note 108 for each chord 106. Identification of root notes 108 of a melody 102 is important (and in some embodiments may be critical) to ensure harmony between the melody 102 and the embedded gamma wave sounds. In an embodiment, chord sequences 104 are identified first followed by the corresponding chords 106 and root notes 108. For instance, the song “Under Your Scars” by Godsmack comprises three chord sequences—(1) Bm, A, G, A; (2) Bm, A, G, F #; and (3) Bm, D, E, G, F #. The first chord sequence is comprised of the chords B minor, A major, and G major. Chord G major has a root note of G. In alternative embodiments, chord sequences 104, chords 106, and root notes 108 may be identified in any order. In a preferred embodiment, as many of the chord sequences, chords, and root notes for such chords are identified as possible, and optimally all chord sequences, all chords in each chord sequence, and all root notes are identified. In an alternative embodiment, one or more chord sequences 104, chords 106, and root notes 108 may not be identified or omitted altogether in the parsing process 1100. In an alternative embodiment, notes (not shown) other than root notes 108 are identified, such as, and not of limitation, third, fifth, seventh, ninth, eleventh, thirteenth, and the like. The parsing process 1100 comprises steps which may be known to those of skill in the art. In alternative embodiments (not shown), the parsing process 1100 may comprise different or additional steps to identify the chord sequences 104, chords 106, root notes 108, and other notes of a melody.

After chord sequences 104, chords 106, and root notes 108 are identified, a gamma-frequency selection process 1200 may be performed. As illustrated in block 1200, a gamma-frequency selection process 1200 comprises selecting a frequency 110 of a gamma wave and embedding 112 the frequency in the melody 102. For a detailed view of the different processes used to select the frequency 110 of a Gamma wave and embed 112 the frequency in the melody 102, refer to FIG. 2. A preferred embodiment of selecting a frequency 110 of a gamma wave comprises selecting a frequency 110 that corresponds to the root note 108 identified. An alternative embodiment of selecting a frequency 110 of a gamma wave comprises selecting a frequency 110 that corresponds to any note (not shown) other than the root note 108. Another alternative embodiment of selecting a frequency 110 of a gamma wave comprises selecting a frequency 110 that best addresses the mental health or neurological condition being addressed. Once the frequency 110 of a gamma wave is selected, the frequency 110 is embedded 112 into the melody 102.

After the frequency 110 of a gamma wave is selected and embedded 112 into the melody 102, an amplitude determination step 114 may be performed to determine the amplitude level 114 of the frequency 110 of a Gamma wave. The decibel level is selected to be sensed by a listener without interfering with the musical aesthetic. In an embodiment, the amplitude level 114 of the frequency 110 of a gamma wave is .1-10 decibels higher than the melody's 102 volume. In other embodiments the decibel level of the gamma wave sound may be 0.5-5 decibels above the melody's volume. In alternative preferred embodiments, the amplitude level 114 of the frequency 110 of the gamma wave is two to four decibels higher than the melody's 102 volume, and optimally three decibels higher than the melody's 102 volume. An approximately three decibel difference does not interfere with the original musical intention of a melody 102 and still provides the same level of mental health benefits as traditional audio gamma-frequency stimulation methods. Identification of the correct amplitude level 114 is important (and in some embodiments may be critical) to ensure the gamma wave sounds won't interfere with the melody 102 but still provide optimal health benefits. In alternative embodiments, a decibel difference higher or lower than the range of 0.1-10 decibels may be used. Once all the selected frequencies 110 are embedded into the melody at specific amplitudes 114, the melody 102 and the resulting set of gamma wave sounds are combined 116 into one computer-readable music file 118 for a user to listen to and enjoy.

Referring now to FIG. 2, a block diagram detailing the steps of the gamma-frequency selection process 1200 is shown. As illustrated in block 1210, a preferred embodiment of selecting a frequency 110 of a gamma wave comprises selecting a frequency 110 that corresponds to the root note 108 identified, root note-to-frequency selection 1211. The melody 102 may comprise a single root note or a plurality of root notes. In a preferred embodiment, a frequency 110 is selected for every root note 108 identified in the melody 102. In other words, the root note-to-frequency selection step 1211 may be repeated to select multiple frequencies of gamma waves depending on how many root notes 108 are identified in the melody 102. In an alternative embodiment, a frequency 110 is not selected for every root note 108 identified in the melody. Selection of a frequency 110 is preferably intended to ensure harmony between the melody and the resulting set of gamma-frequency sounds. The table below outlines a preferred but exemplary embodiment of root note-to-frequency selection 1211, specifying which frequency 110 is selected for the identified root note 108. The frequencies identified for each root note in the table below are preferred to ensure harmony between the melody 102 and the embedded gamma wave sounds. While the table represents a preferred embodiment of root note-to-frequency selection 1211, it will be understood that other embodiments may comprise selecting different frequencies for each root note. In such alternative embodiments, the selection of frequencies for root notes may vary, but the process of associating a particular frequency with a root note to ensure harmony remains the same.

Once a frequency is selected, the frequency 110 is embedded at the corresponding chord 106. For instance, chord G, which is found in the first chord sequence of the song “Under Your Scars” by Godsmack, has a root note of G. A gamma wave with a frequency of 49 Hz is embedded at the location of chord G to ensure harmony between the chord and the gamma wave sound. In a preferred embodiment, wherein a melody comprises a plurality of root notes, multiple frequencies are selected and embedded at the corresponding chord locations, and optimally, frequencies are selected for every root note identified and embedded at every corresponding chord location. In such embodiments, approximately 90% of the melody is embedded with gamma waves and optimally 100% of the melody is embedded with gamma waves. For instance, the song “Under Your Scars” by Godsmack comprises three root notes-B, A, and G. In this example, gamma waves with frequencies of 61.7 Hz, 55 Hz, and 49 Hz, respectively, are selected and embedded at the corresponding chord locations throughout the entirety of the melody. In an alternative embodiment, frequencies are not selected for every root note identified or not embedded at every corresponding chord location. In such embodiments, less than 90% of the melody is embedded with gamma waves, for example, though not of limitation, to achieve an aesthetically pleasing result that is still therapeutically effective. For instance, embedding a gamma wave in the guitar intro of the song “Paint it Black” by the Rolling Stones may be disruptive or destroy the integrity of the guitar intro; thus, to achieve an aesthetically pleasing end result, gamma waves may be excluded from that portion of the song. As illustrated in block 1220, an alternative embodiment of selecting a frequency 110 of a gamma wave comprises selecting a frequency 110 that corresponds to notes other than the root note 108, note-to-frequency selection 1221. Once a frequency is selected, the frequency 110 of the gamma wave is embedded 1222 at the corresponding chord 106, note, chord sequence 104, or the like. As illustrated in block 1230, an alternative embodiment of selecting a frequency 110 of a gamma wave comprises selecting a frequency 110 that best addresses the mental health or neurological condition being treated, condition-to-frequency selection 1231. For instance, it is well published that gamma-frequency auditory stimulation delivered at 40 Hz significantly improves cognition and found to improve multiple biomarkers of Alzheimer's. Once a frequency that best addresses the condition being treated is selected, the frequency is embedded 1232 into the melody in a manner that does not disrupt the melody's harmony. In alternative embodiments (not shown), the gamma-frequency selection process 1200 may comprise different or additional processes and steps for selecting a frequency 110 of a gamma wave.

An exemplary association between root note and gamma wave frequency is shown below in Table 1:

TABLE 1

| Root Note | Frequency |
|---|---|
| C-1 | 32.7 Hz |
| C#-1 | 34.6 Hz |
| D-1 | 36.7 Hz |
| D#-1 | 38.9 Hz |
| E-1 | 41.2 Hz |
| F-1 | 43.6 Hz |
| F#-1 | 46.2 Hz |
| G-1 | 49.0 Hz |
| G#-1 | 51.9 Hz |
| A-1 | 55.0 Hz |
| A#-1 | 58.3 Hz |
| B-1 | 61.7 Hz |
| C 0 | 65.4 Hz |

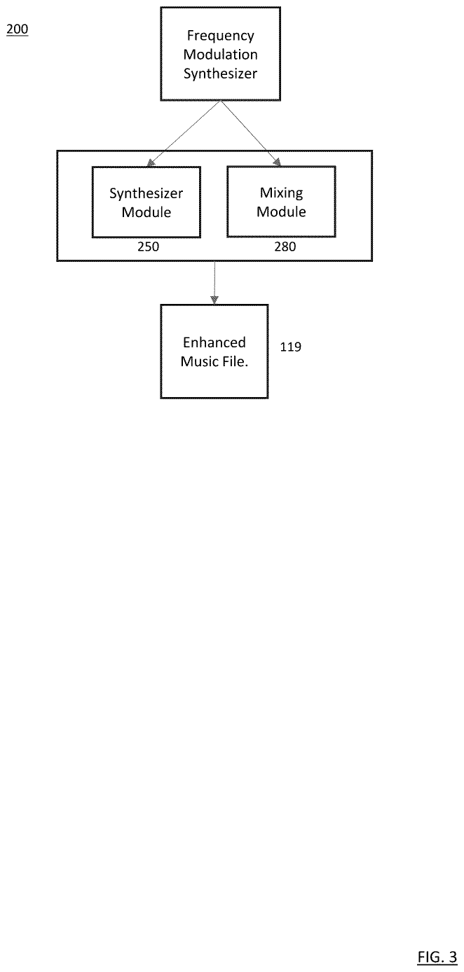

Referring now to FIG. 3, a block diagram is shown of an embedding system 200 according to an embodiment of the invention. Embedding system 200 is preferably a Frequency Modulation Synthesizer, specifically Ableton Live. Embedding system 200 comprises several modules such as, without limitation, synthesizer module 250, mixing module 280, and the like. In alternative embodiments, embedding system 200 may comprise a system or device other than a Frequency Modulation Synthesizer, such as a laptop, DJ controller and mixer, music production machine, and the like comprising capabilities, applications, and the like, capable of performing the functions and achieving the results described herein. For instance, and not of limitation, embedding system 200 may be a computer comprising a frequency modulation synthesizer application other than Ableton Live wherein the application performs the same or similar functions and achieves the intended enhanced music file 119 described herein.

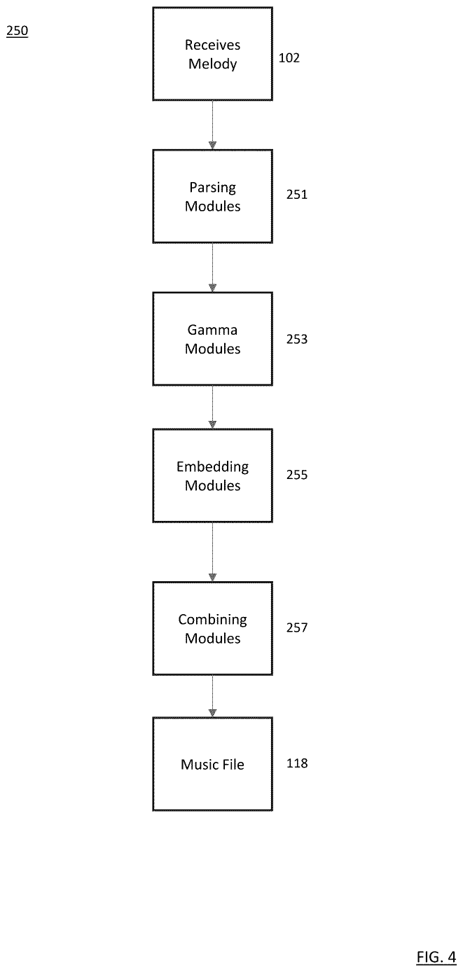

Referring now to FIG. 4, a block diagram of the synthesizer module 250 is shown together with its interaction with the melody 102 according to an embodiment of the invention. The primary function of the synthesizer module 250 is to process the melody 102 and allow the user to parse and organize the melody 102 into different sections which is essential for executing the preferred method 100. The synthesizer module 250 facilitates the implementation of the preferred method 100 by allowing a user to accurately detect chord sequences 104, chords 106, root notes 108, and other notes of a melody which serve as input for selecting a frequency 110 of a gamma wave. Further, the synthesizer module 250 allows the user to create gamma wave sounds and embed gamma wave sounds into the melody. As depicted, the synthesizer module 250 comprises parsing modules 251, gamma modules 253, embedding modules 255, and combine files modules 257.

Synthesizer module 250 receives a melody 102 which may be retrieved from a RAM, USB, optical disk, SDs, disk storage, ROM, or the like. Synthesizer module 250 comprises one or more parsing modules 251 which allows the user to parse the melody into different sections. Parsing the melody 102 into different sections allows for easier identification of chord sequences 104, chords 106, and root notes 108. In an alternative embodiment, melody parsing can be performed manually by a person skilled in the art without requiring a synthesizer module 250 comprising parsing modules 251 or the like.

Synthesizer module 250 comprises one or more gamma modules 253 to allow the user to create gamma wave sounds at a particular frequency 110. For instance, gamma module 253 may comprise a gamma synthesizer programmed with pre-chosen gamma-frequency sounds for a user to select sounds which correspond to the root note of the melody.

Synthesizer module 250 comprises one or more embedding modules 255 to allow the user to embed the frequency 110 into the melody 102. For instance, where the user has access to a studio isolated multi-track recording, the user can use specific embedding modules 255 to artistically blend the gamma wave into the track. In instances where the user only has access to the master recording, the user can use other embedding modules 255 to blend the gamma wave into the track. The synthesizer module 250 comprises one or more combine files modules 257 to allow the user to combine the melody and the gamma-frequency sounds into one music file 118. The output of the synthesizer module 250 is a single computer-readable music file 118, such as, though not of limitation, a .wav audio file, comprising both the melody and corresponding gamma frequencies for a user to listen to and enjoy.

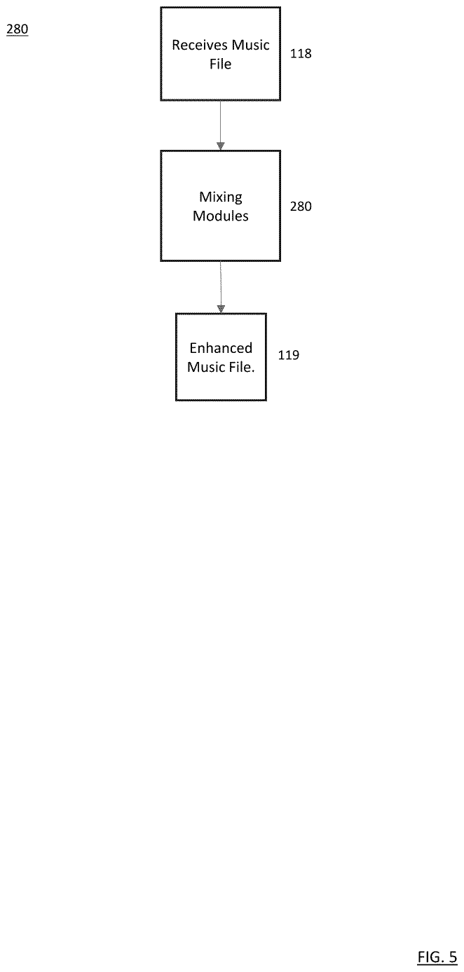

Referring now to FIG. 5, a block diagram of mixing module 280 is shown together with its interaction with the music file 118 according to an embodiment of the invention. Mixing module 280 receives a music file 118 and comprises one or more mixing modules to allow the user to enhance the music file 118 further and prepare the audio file 118 for different delivery methods such as, though not of limitation, virtual reality, audio, visual, audio-visual, and the like. The resulting output of the mixing module 280 is an enhanced computer-readable music file 119, prepared for a specific delivery method, comprising both the melody and corresponding gamma frequencies for a user to listen to and enjoy. Repositories of such songs can be maintained in a database that may be accessible for free or for a fee, at a specific therapeutic location or on-line, or for distribution on a transferrable medium.

The treatment of an individual with a melody embedded with gamma-frequency sounds according to embodiments of the invention can occur in a variety of ways. For instance, though not of limitation, the individual can access the melody through an audio-playing device such as a phone or mp3 player, or can stream the inventive melody directly from an on-line repository of such songs, or experience the melody in a designated therapeutic environment such as a therapist's office, wellness center, spa or treatment facility. Such treatments can be delivered in a variety of settings with fine-tuned sensory accompaniments, such as massages, or visual stimulation, either by projection, ambience, or through direct stimulation (e.g., virtual reality headsets or the like).

Although the present invention and its advantages have been described in detail, it should be understood that various changes, substitutions and alterations can be made herein without departing from the spirit and scope of the invention as defined in the appended claims.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed:

1. A method for incorporating gamma-frequency sound waves into a melody, comprising:
identifying a chord sequence of the melody, the melody comprising a plurality of chord sequences;
identifying a chord of the chord sequence;
identifying a root note for the chord;
identifying an amplitude level of the chord sequence;
selecting a gamma wave having a selected gamma wave frequency of between 15 Hz and 80 Hz, the selected gamma wave frequency selected to correspond to the root note;
embedding the selected gamma wave over the chord sequence at a gamma wave amplitude of .1 to 10 decibels above the chord sequence amplitude level; and
repeating the above steps for one or more additional chord sequences to produce a gamma wave embedded melody.

2. A computer-readable medium, having computer-readable program code for music embodied therewith, comprising:
a first melody comprising a series of musical notes,
a second melody comprising repeating tones in the range of 15 Hz to 80 Hz,
wherein the first melody and the second melody are merged,
wherein the first melody and second melody are merged to have a less than 3 decibel discrepancy.

3. A method for treating a mood disorder in people comprising:
identifying and providing a melody aesthetically pleasing to one or more individuals to be treated;
identifying a chord sequence of the melody, the melody comprising a plurality of chord sequences;
identifying a chord of the chord sequence;
identifying a root note for the chord;
identifying an amplitude level of the chord sequence;
selecting a gamma wave having a selected gamma wave frequency of between 15 Hz and 80 Hz, the selected gamma wave frequency selected to correspond to the root note;
embedding the selected gamma wave over the chord sequence at a gamma wave amplitude of .1 to 10 decibels above the chord sequence amplitude level;
repeating the above steps for one or more additional chord sequences to produce a gamma wave embedded melody; and
providing or playing the gamma wave embedded melody to the individual.

4. The method of claim 1, wherein the melody is a song.

5. The method of claim 1, wherein the melody is a musical composition.

6. The method of claim 1, wherein the selected gamma wave is embedded over the chord sequence at a gamma wave amplitude of 3 decibels above the chord sequence amplitude level.

7. The method of claim 1, further comprising a smooth transition between each gamma wave in the melody.

8. The method of claim 1, wherein a root note is associated with a gamma frequency selected from the group consisting of: 32.7 Hz for the root note C-1, 34.6 Hz for the root note C#-1, 36.7 Hz for the root note D-1, 38.9 Hz for the root note D #-1, 41.2 Hz for the root note E-1, 43.6 Hz for the root note F-1, 46.2 Hz for the root note F #-1, 49.0 Hz for the root note G-1, 51.9 Hz for the root note G #-1, 55.0 Hz for the root note A-1, 58.3 Hz for the root note A #-1, 61.7 Hz for the root note B-1, and 65.4 Hz for the root note C 0.

9. The method of claim 3, wherein the gamma wave embedded melody to the individual is delivered audibly.

10. The method of claim 3, wherein the gamma wave embedded melody to the individual is delivered in a virtual environment with audio components.

11. The method of claim 3, wherein the gamma wave embedded melody to the individual is delivered in a virtual environment with both visual and audio components.

12. The method of claim 3, wherein the gamma wave embedded melody to the individual is delivered simultaneously in visual and audio formats.
